(19) United States
(12) Patent Application Publication
(10) Pub. No.: US 2025/0128073 A1
(43) Pub. Date: Apr. 24, 2025

(54) ACTIVE ELECTRICAL SYSTEM AND METHOD FOR OPERATING AN ACTIVE ELECTRICAL SYSTEM

(71) Applicant: BIOTRONIK SE & Co. KG, Berlin (DE)

(72) Inventors: Ingo WEISS, Berlin (DE); René FISCHER, Berlin (DE)

(73) Assignee: BIOTRONIK SE & Co. KG, Berlin (DE)

(21) Appl. No.: 18/834,323

(22) PCT Filed: Feb. 7, 2023
(86) PCT No.: PCT/EP2023/052987

§ 371 Date: Jul. 30, 2024

(30) Foreign Application Priority Data
Feb. 11, 2022 (EP) 22156232.5

(51) Int. Cl. A61N 1/365; A61B 5/0245; A61B 5/349; A61N 1/02; A61N 1/37; A61N 1/39

(52) U.S. Cl. CPC A61N 1/365; A61B 5/0245; A61B 5/349; A61N 1/025; A61N 1/39622; A61N 1/3706

(57) ABSTRACT

An active electrical system comprising implantable components and an external programming unit, the system further comprising an algorithm for classifying the sensed electrical cardiac signals, and a control unit configured to drive the pulse delivery unit in a first mode to deliver a predetermined number of pulses at repetition rates greater than or equal to a threshold value, and to drive the pulse delivery unit in a second mode to deliver a predetermined maximum number of pulses at repetition rates below the threshold value, and wherein the control unit in the second mode is configured to switch to the first mode or terminate pulse delivery depending on a classification result of the algorithm.

### CROSS-REFERENCE TO RELATED APPLICATIONS

This application is the United States National Phase under 35 U.S.C. § 371 of PCT International Patent Application No. PCT/EP2023/052987, filed on Feb. 7, 2023, which claims the benefit of European Patent Application No. 22156232.5, filed on Feb. 11, 2022, the disclosures of which are hereby incorporated by reference herein in their entireties.

### TECHNICAL FIELD

The present invention relates to an active electrical system. Furthermore, the present invention relates to a method for operating an active electrical system.

### BACKGROUND

Conventional subcutaneous ICD systems employ VVI pacing at low pacing rates. VVI pacing consists of resetting a counter or a timing unit to a start value after a detected intrinsic cardiac signal or a stimulus, respectively. When the counter or the waiting time reaches a threshold value, a stimulus is triggered. Detection of an intrinsic cardiac signal may be performed by comparison with a threshold value. For example, the signal amplitude or another morphological metric is compared to such a threshold value. Whether the stimulus is successful and triggers a cardiac signal is not relevant in this context.

Furthermore, there are problems posed by afterpotentials at the electrodes, especially as a result of high-intensity stimuli. These overlay a sensed signal waveform. The signal can even exceed a measuring range limit. As long as this is the case, any signal information is lost. The saturation of amplifiers and the overflow of filters usually caused by this leads to a condition that no appropriate sensing of the physiological signals can take place even for a longer time.

A blanking window is intended to prevent/reduce these causes. For this purpose, it must be considerably longer, especially for stimuli of high intensity, so that sensing is blinded, i.e., not performed for a predetermined period of time. The intrinsic heart signal, because it is still below the threshold value, is not detectable due to the afterpotential which has not completely decayed even after this and the stimulus would be delivered erroneously. This must therefore be scheduled much later so that there is still the possibility of sensing the intrinsic cardiac signal and suppressing the stimulus. The maximum possible stimulation rate is therefore very limited and may not be sufficient for hemodynamic care.

The present disclosure is directed toward overcoming one or more of the above-mentioned problems, though not necessarily limited to embodiments that do.

### SUMMARY

It is therefore an object of the present invention to provide an improved active electrical system capable of providing improved post-shock stimulation from the beginning with increased hemodynamic output and still reliably detecting a return of an intrinsic heart rhythm.

At least the object is solved by an active electrical system having the features of claim 1. In addition, at least the object is solved by a method for operating an active electrical system having the features of claim 8.

Further developments and advantageous embodiments are defined in the dependent claims.

The present invention provides an active electrical system comprising implantable components and an external programming unit. The system comprises at least two electrode poles forming at least one electrode pair configured to provide energy delivery and sensing.

Furthermore, the system comprises a pulse delivery unit for generating electrical pulses and delivery via the at least one electrode pair and a sensing unit for sensing electrical cardiac signals via the at least one electrode pair within a predetermined time period.

Moreover, the system comprises an algorithm for classifying the sensed electrical cardiac signals, and a control unit configured to drive the pulse delivery unit in a first mode to deliver a predetermined number of pulses at repetition rates greater than or equal to a threshold value, and to drive the pulse delivery unit in a second mode to deliver a predetermined maximum number of pulses at repetition rates below the threshold value, and wherein the control unit in the second mode is configured to switch to the first mode or terminate pulse delivery depending on a classification result of the algorithm.

The present invention further provides a computer-implemented method for operating an active electrical system comprising implantable components and an external programming unit.

The method comprises providing energy delivery and sensing by means of at least two electrode poles forming at least one electrode pair and generating electrical pulses and delivering said electrical pulses via the at least one electrode pair by means of a pulse delivery unit.

Furthermore, the method comprises sensing electrical cardiac signals via the at least one electrode pair within a predetermined time period by means of a sensing unit, classifying the sensed electrical cardiac signals by means of an algorithm, and driving the pulse delivery unit in a first mode to deliver a predetermined number of pulses at repetition rates greater than or equal to a threshold value, and driving the pulse delivery unit in a second mode to deliver a predetermined maximum number of pulses at repetition rates below the threshold value by means of a control unit, and wherein the control unit in the second mode switches to the first mode or terminates pulse delivery depending on a classification result of the algorithm.

It is an idea of the present invention to provide post-shock stimulation with increased hemodynamic output and reliable detection of a return of an intrinsic heart rhythm. Moreover, prompt redetection of an unsuccessfully treated malignant rhythm and/or malignant rhythm resuming during post-shock stimulation is provided.

The present invention further enables energy delivery and appropriate, fast/prompt sensing on the same vector, i.e., on the same pair of electrodes. At least this object is solved especially for non-transvenous defibrillators.

According to an aspect of the present invention, the active electrical system is configured to provide post-shock stimulation in implantable defibrillators, in particular in non-transvenous defibrillators.

According to a further aspect of the present invention, the algorithm is configured to classify a total and/or ventricular cardiac arrest, an intrinsic non-malignant rhythm, an intrinsic malignant rhythm and/or technical or physiological signal disturbances.

According to a further aspect of the present invention, the algorithm is configured to determine an occurrence of the cardiac arrest by comparing a signal range and/or a signal energy with a predetermined threshold value.

The term signal range is to be understood as the difference max-min within a given time window. The time window can also be understood to be very large (quasi infinite). Then it means the difference of the extreme values that are to be expected, e.g., if for intrinsic signals the difference max-min is +/−5 mV, the signal range would be 10 mV.

According to a further aspect of the present invention, the algorithm is configured to detect and classify an intrinsic heart rhythm by evaluating cardiac events, in particular a trigger timing, morphological features of a signal curve around a cardiac event, in particular an area under the signal curve, a peak-to-peak time interval, a jagged difference, and signal blocks, in particular metrics of block features. To get the jagged difference of a signal, the signal peaks (peak amplitudes) are searched for between zero crossings and a vector is formed from the differences of the amplitude values of neighboring peak amplitudes.

The term “jagged difference” is understood to mean at least one of the following metrics. Based on a signal with a series of local extrema, said local extrema appear as jags. The peaks are the local extrema (i.e., maxima and minima). Local maxima can also lie below the zero line, and local minima can lie above the zero line. Between two points is not necessarily a zero crossing.

First metric: According to the present invention a first metric is defined as a vector which lists the amplitude values of these extremes chronologically. It is thus inevitable that every second entry is a max and every other second entry is a min. With the additional information what the first entry is (max or min) it is unambiguous. The signal morphology is hence roughly sketched. In addition, the corresponding time points can be stored in a second vector.

Second metric: Only the differences of adjacent extreme values are stored. So max to min then min to max etc. Optionally, additionally the time points can be stored. If only the absolute values of it are taken, these are then locally seen as jagged strokes.

Third metric: Only max-min but not min-max, then again max-min are stored, i.e., only every second value of the second metric. Optionally, additionally the time points are stored.

Fourth metric: Only the differences max-max are stored, i.e., only the max are retrieved from the first metric and a difference vector is formed. Optionally, additionally the time points are stored.

Fifth metric: Only the differences min-min are stored, i.e., only the min are retrieved from the first metric and a difference vector is formed. Optionally, additionally the time points are stored.

According to a further aspect of the present invention, the algorithm is configured to determine and distinguish between malignant and non-malignant heart rhythms and/or signal features.

According to a further aspect of the present invention, the algorithm is configured to detect signal perturbations by evaluating metrics and/or comparing with a threshold value in the time domain, in particular counting zero crossings, and/or in the frequency domain, in particular determining a signal energy in specific frequency bands.

According to a further aspect of the present invention, the predetermined time period within which the algorithm classifies the sensed electrical cardiac signals begins after a blanking window of programmable duration and with programmable starting time relative to a delivered electrical pulse.

According to a further aspect of the present invention, the duration and starting time of the blanking window is adjusted depending on the delivered electrical pulses and their frequency.

According to a further aspect of the present invention, during the blanking window, a high impedance isolation of the sensing unit, a delivery of a compensating pulse to minimize an afterpotential, short-circuiting of the electrode poles sensing thereafter, desaturating a sensing amplifier and/or setting the cardiac signal to a fixed value is performed.

According to a further aspect of the present invention, the threshold value, in particular a programmable rate threshold, by which the control unit switches between the first mode and the second mode is in a range between 25 and 80 bpm, wherein a higher rate during the first mode is higher by at least a predetermined value relative to the threshold value, in particular 5 bpm, 10 bpm or 20 bpm higher than the threshold value, and wherein a lower rate during the second mode is lower by a predetermined value relative to the threshold value, in particular 5 bpm, 10 bpm or 20 bpm lower than the threshold value. The actual rates in the modes can optionally be assigned a programmable heart rate variability.

According to a further aspect of the present invention, the number of pulses in the first mode is programmable between one to ten and/or a maximum duration remaining in the first mode, and wherein the number of pulses in the second mode is programmable between zero and five and/or a maximum duration remaining in the second mode.

According to a further aspect of the present invention, the control unit starts after a predetermined delay after a previous event, in particular a shock, to set the pulse delivery unit initially into the first mode, and wherein after a predetermined delay following a previous event, the control unit starts to set the pulse delivery unit to the second mode.

According to a further aspect of the present invention, the control unit switches to the first mode when a cardiac arrest, in particular an AV block or a heart rate lower than the pacing rate, is detected and/or confirmed in at least a second pulse interval of the second mode, and wherein the control unit terminates pulse delivery when an intrinsic cardiac rhythm is detected in the second mode.

Moreover, the control unit extends a time in the first mode when a signal fault is detected. The time extension is a programmable additional number of pulses or a duration. The control unit further selects an alternative vector for sensing, if a signal disturbance is detected.

In addition, the control unit optionally initiates recording/storage of a subcutaneous ECG due to programmable triggering during post-shock stimulation (e.g., upon re-detection of a malignant rhythm during post-shock stimulation, reaching the maximum programmed post-shock stimulation duration without detected intrinsic rhythm and repeated noise triggering during post-shock stimulation).

The herein described features of active electrical system are also disclosed for the method for operating the active electrical system and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a block diagram of an active electrical system according to a preferred embodiment of the present invention:

FIG. 2a shows a flowchart of the method for operating the active electrical system according to the preferred embodiment of the present invention;

FIG. 2b shows a flowchart of the method for operating the active electrical system according to the preferred embodiment of the present invention;

FIG. 3 shows a signal curve of the method for operating the active electrical system according to the preferred embodiment of the present invention; and

FIG. 4 shows a flowchart of the method for operating the active electrical system according to the preferred embodiment of the present invention.

### DETAILED DESCRIPTION

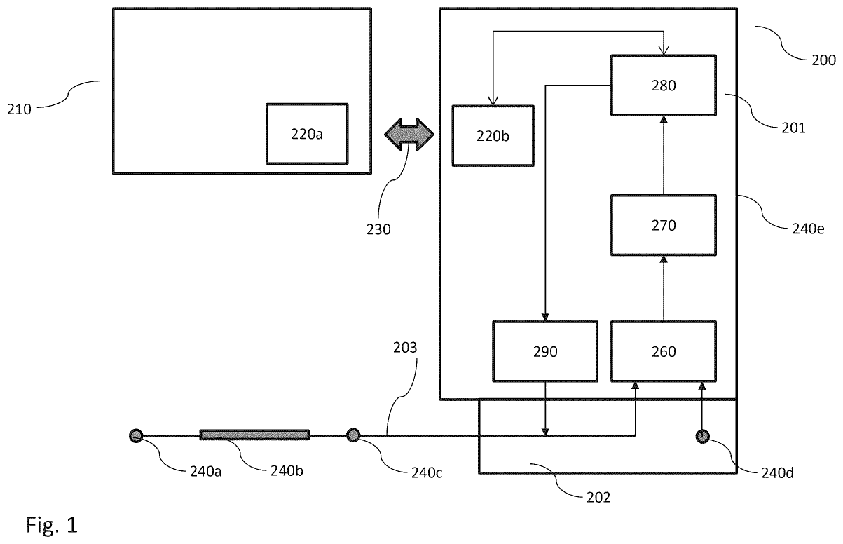

FIG. 1 shows a block diagram of an active electrical system according to a preferred embodiment of the present invention. The system comprises at least two electrode poles 240a-e forming at least one electrode pair configured to provide energy delivery and sensing.

Furthermore, the system comprises a pulse delivery unit 290 for generating electrical pulses and delivery via the at least one electrode pair and a sensing unit 260 for sensing electrical cardiac signals via the at least one electrode pair within a predetermined time period.

Moreover, the system comprises an algorithm 270 for classifying the sensed electrical cardiac signals, and a control unit 280 configured to drive the pulse delivery unit 290 in a first mode 301 to deliver a predetermined number of pulses at repetition rates greater than or equal to a threshold value, and to drive the pulse delivery unit 290 in a second mode 302 to deliver a predetermined maximum number of pulses at repetition rates below the threshold value, and wherein the control unit 280 in the second mode 302 is configured to switch to the first mode or terminate pulse delivery depending on a classification result of the algorithm 270. Further, the implanted unit and the programmer are configured to communicate 230 via devices 220a and 220b.

The implantable system 200 further comprises a device 201 and electrode leads 203 optionally connected thereto. The electrode poles are also located on the electrode lead and optionally partially on the device, e.g., also on the header 202.

The electrode poles are formed as domes 240a, rings 240c, coils 240b, housing 240e or parts thereof, or are attached to insulated parts of the housing 240d, having a raised, flat or recessed design.

The pulse delivery unit 290 comprises capacitors, transformers and electronic switches, e.g., IGBT, AGT, etc. The sensing unit 260 comprises an AD converter, analog and/or digital filters, a memory and means for offset compensation.

Moreover, the same electrode poles form both an energy output vector and a sensing vector. The vectors, i.e., the electrode pair, for energy output and sensing share at least one electrode pole. In addition, the vectors of energy output and sensing form disjoint sets.

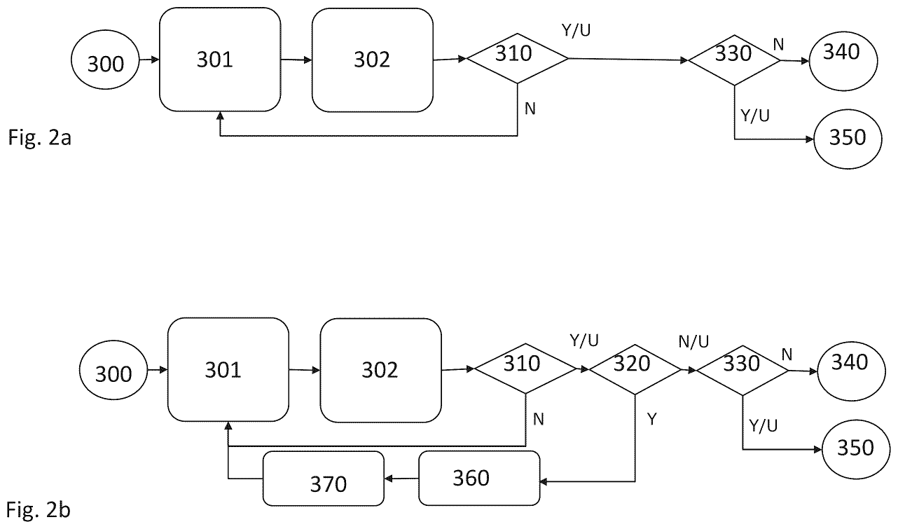

FIG. 2a shows a flowchart of the method for operating the active electrical system according to the preferred embodiment of the present invention, in particular a more concrete embodiment of the present invention for the control unit 280. FIG. 2a shows the control unit 280 without consideration of disturbances.

Said control unit 280 is configured to switch between the first mode and the second mode or termination of pulse delivery, where the first mode 301 therapy mode delivers a programmable number of stimuli at repetition rates greater than or equal to a programmable rate or for a programmable duration and the second mode 302 listening mode with therapy support delivers a programmable maximum number of stimuli or programmable maximum duration at repetition rates below this rate and in the second mode, depending on the classification result, it switches to the first mode or terminates the pulse delivery.

In step 310 it is determined whether an intrinsic heart rhythm is detected. In step 330 it is determined whether a malignant rhythm is detected. At step 340, post-shock stimulation is ended when an intrinsic rhythm is present. At step 350, post-shock stimulation is ended when a malignant rhythm is redetected.

FIG. 2b shows a flowchart of the method for operating the active electrical system according to the preferred embodiment of the present invention. FIG. 2b shows the control unit 280 with consideration of disturbances.

According to the present invention, said method can be started at 301 or 302, wherein 300 designates start post shock stimulation. 301 designates the first mode with parameters pulse, duration, frequency and blanking. Step 302 designates the second mode with parameters pulse, duration, frequency and blanking. Blanking is preferably set automatically depending on a post-shock stimulation frequency.

In step 310 it is determined whether an intrinsic heart rhythm is detected. In step 320 it is determined whether noise is detected. In step 330 it is determined whether a malignant rhythm is detected. At step 340, post-shock stimulation is ended, an intrinsic rhythm is present.

At step 350, post-shock stimulation is ended when a malignant rhythm is redetected. Step 360 designates a switch to an alternative lead and step 370 designates an increase of a time period. Y designates yes, N designates no and U designates unknown.

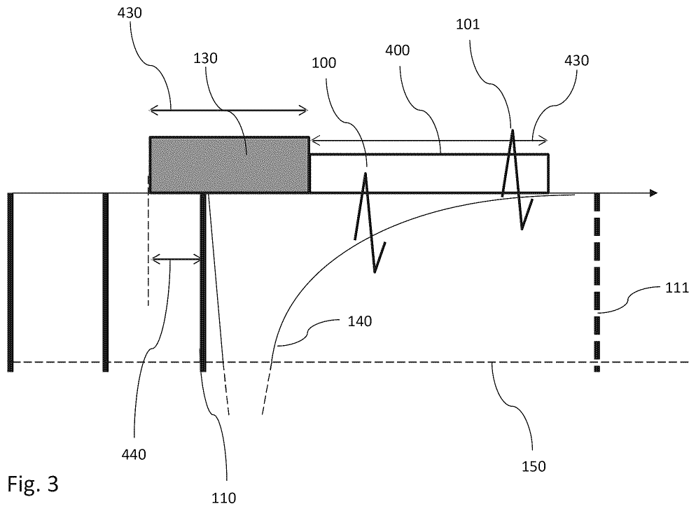

FIG. 3 shows a signal curve of the method for operating the active electrical system according to the preferred embodiment of the present invention. FIG. 3 shows the programmable time window 400 within which the algorithm classifies sensed electrical heart signals 101. 100 designates the intrinsic heart signal.

This begins after a previously switched blanking window 130 of programmable duration 430 and with programmable start time point 440 relative to one of the emitted electrical pulses 110 in particular, this blanking window can also begin before this. Another pulse 111 would follow if the electrical heart signal 101 had not been present or sensed.

140 designates the afterpotential at the electrodes, especially as a result of stimuli 110 of high intensity. These overlay the sensed signal waveform. The signal can even exceed the measuring range limit 150.

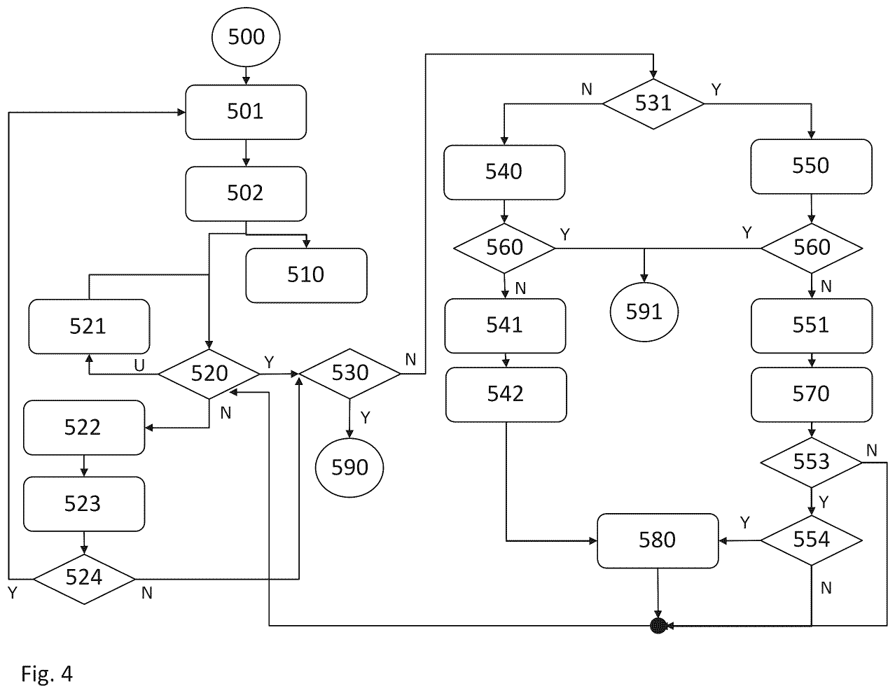

FIG. 4 shows a flowchart of the method for operating the active electrical system according to the preferred embodiment of the present invention. FIG. 4 shows a more concrete embodiment of the solution according to the present invention for the control unit 280.

At step 500, confirmation to deliver an initial therapy shock (malignant rhythm detected is provided. After delivery of the initial therapy shock 501 and waiting for an initial blanking time 502, the heart rate estimation is restarted 510. If it is unclear whether the malignant rhythm is still present 520, an additional assessment phase is inserted once 521. If the malignant rhythm is confirmed, charging is restarted 522. After the charging voltage 523 has been reached, a check is made to see whether the malignant rhythm is still present 524. If yes, this new therapy shock is again the starting point for the procedure already described.

If not, it is checked whether intrinsic rhythm can be detected above the stimulation frequency 530. This is also the subsequent step if the malignant rhythm terminated by the previous therapy shock was detected in 520. If intrinsic rhythm can be detected, post-shock stimulation is terminated, or not started at all 590. If no intrinsic rhythm is detected, the post-shock stimulation is started. At step 531, it is determined whether post-shock stimulation is parameterized as V00.

This allows the use of the “main sensing algorithm”, which can be used without the presence of stimulation artifacts, to evaluate the signal for the presence of malignant rhythm. A special “post-shock stimulation sensing algorithm” is used if the user prefers a classical post-shock stimulation in the form of V00 stimulation 550. The alternative post-shock stimulation variant is referred to as periodic 540. At step 560, it is determined whether the maximum total number of stimuli or the maximum effective stimulation time has been reached.

In this alternative post-shock stimulation variant, a number of stimuli are delivered at a predetermined frequency 541. Subsequently, no stimuli are delivered for a time, allowing the main algorithm to perform signal evaluation. Moreover, at step 542, waiting for classification phase of predetermined length occurs. At step 580 classification of the signal with the main sensing algorithm occurs. At step 551 delivery of a single stimulus is performed an at step 570 classification of the signal with the algorithm occurs.

Also, in the classical post-shock stimulation an automatic switching to the main algorithm is provided, if an intrinsic rhythm is detected again 553, 554. With the result of the evaluation the next cycle starts again at 520. Furthermore, at step 591, post-shock stimulation is ended due to a very slow rhythm and a maximum effective stimulation time has been reached.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

100 intrinsic heart signal
101 electrical heart signals
110 electrical pulses
111 pulse
130 blanking window
140 afterpotential
150 measuring range limit
200 implantable components
201 device
202 header
203 electrode leads
210 programming unit
220a, 220b devices
240a domes
240b coils
240c rings
240d parts of the housing
240e housing
260 sensing unit
270 algorithm
280 control unit
290 pulse delivery unit
300 Post Shock Stimulation
301 first mode
302 second mode
310 determining step
320 determining step
330 determining step
340 post-shock stimulation end
350 post-shock stimulation end
360 switch to an alternative lead
370 increase of a time period
400 programmable time window
430 programmable duration
440 start time point
500 confirmation step
501 initial therapy shock
502 initial blanking time
510 Restart
520 detection step
521 assessment phase
522 Restart
523 charging voltage
524 check
530 check
531 determining step
540 alternative post-shock stimulation variant
541 frequency
542 waiting step
550 V00 stimulation
551 delivery step
553 detecting step
554 detecting step
560 determining step
570 classification
580 classification
590 termination step
591 post-shock stimulation end
N No
U Unknown
Y Yes

1. Active electrical system comprising implantable components and an external programming unit, the system further comprising:
at least two electrode poles forming at least one electrode pair configured to provide energy delivery and sensing;
a pulse delivery unit for generating electrical pulses and delivery via the at least one electrode pair;
a sensing unit for sensing electrical cardiac signals via the at least one electrode pair within a predetermined time period;
an algorithm for classifying the sensed electrical cardiac signals; and
a control unit configured to drive the pulse delivery unit in a first mode to deliver a predetermined number of pulses at repetition rates greater than or equal to a threshold value, and to drive the pulse delivery unit in a second mode to deliver a predetermined maximum number of pulses at repetition rates below the threshold value, and wherein the control unit in the second mode is configured to switch to the first mode or terminate pulse delivery depending on a classification result of the algorithm.

2. Active electrical system of claim 1, wherein the active electrical system is configured to provide post-shock stimulation in implantable defibrillators, in particular in non-transvenous defibrillators.

3. Active electrical system of claim 1, wherein the algorithm is configured to classify a total and/or ventricular cardiac arrest, an intrinsic non-malignant rhythm, an intrinsic malignant rhythm and/or technical or physiological signal disturbances.

4. Active electrical system of claim 1, wherein the algorithm is configured to determine an occurrence of the cardiac arrest by comparing a signal range and/or a signal energy with a predetermined threshold value.

5. Active electrical system of claim 1, wherein the algorithm is configured to detect and classify an intrinsic heart rhythm by evaluating cardiac events, in particular a trigger timing, morphological features of a signal curve around a cardiac event, in particular an area under the signal curve, a peak-to-peak time interval, a jagged difference, and signal blocks, in particular metrics of block features.

6. Active electrical system of claim 1, wherein the algorithm is configured to determined and distinguish between malignant and non-malignant heart rhythms and/or signal features.

7. Active electrical system of claim 1, wherein the algorithm is configured to detect signal perturbations by evaluating metrics and/or comparing with a threshold value in the time domain, in particular counting zero crossings, and/or in the frequency domain, in particular determining a signal energy in specific frequency bands.

8. Computer-implemented method for operating an active electrical system comprising implantable components and an external programming unit, the method comprising:
providing energy delivery and sensing by means of at least two electrode poles forming at least one electrode pair;
generating electrical pulses and delivering said electrical pulses via the at least one electrode pair by means of a pulse delivery unit;
sensing electrical cardiac signals via the at least one electrode pair within a predetermined time period by means of a sensing unit;
classifying the sensed electrical cardiac signals by means of an algorithm; and
driving the pulse delivery unit in a first mode to deliver a predetermined number of pulses at repetition rates greater than or equal to a threshold value, and driving the pulse delivery unit in a second mode to deliver a predetermined maximum number of pulses at repetition rates below the threshold value by means of a control unit, and wherein the control unit in the second mode switches to the first mode or terminate pulse delivery depending on a classification result of the algorithm.

9. Computer-implemented method of claim 8, wherein the predetermined time period within which the algorithm classifies the sensed electrical cardiac signals begins after a blanking window of programmable duration and with programmable starting time relative to a delivered electrical pulse.

10. Computer-implemented method of claim 9, wherein the duration and starting time of the blanking window is adjusted depending on the delivered electrical pulses and their frequency.

11. Computer-implemented method of claim 8, wherein during the blanking window, a high impedance isolation of the sensing unit, a delivery of a compensating pulse to minimize an afterpotential, short-circuiting of the electrode poles sensing thereafter, desaturating a sensing amplifier and/or setting the cardiac signal to a fixed value is performed.

12. Computer-implemented method of claim 8, wherein the threshold value, in particular a programmable rate threshold, by which the control unit switches between the first mode and the second mode is in a range between 25 and 80 bpm, wherein a higher rate during the first mode is higher by at least a predetermined value relative to the threshold value, in particular 5 bpm, 10 bpm or 20 bpm higher than the threshold value, and wherein a lower rate during the second mode is lower by a predetermined value relative to the threshold value, in particular 5 bpm, 10 bpm or 20 bpm lower than the threshold value.

13. Computer-implemented method of claim 8, wherein the number of pulses in the first mode is programmable between one to ten and/or a maximum duration remaining in the first mode, and wherein the number of pulses in the second mode is programmable between zero and five and/or a maximum duration remaining in the second mode.

14. Computer-implemented method of claim 8, wherein the control unit starts after a predetermined delay after a previous event, in particular a shock, to set the pulse delivery unit initially into the first mode, and wherein after a predetermined delay following a previous event, the control unit starts to set the pulse delivery unit to the second mode.

15. Computer-implemented method of claim 8, wherein the control unit switches to the first mode when a cardiac arrest, in particular an AV block or a heart rate lower than the pacing rate, is detected and/or confirmed in at least a second pulse interval of the second mode, and wherein the control unit terminates pulse delivery when an intrinsic cardiac rhythm is detected in the second mode.
